(19) United States
(12) Patent Application Publication
(10) Pub. No.: US 2025/0339669 A1
(43) Pub. Date: Nov. 6, 2025

(54) BEARING DEVICE FOR A HEART SUPPORT SYSTEM, AND METHOD FOR RINSING A SPACE IN A BEARING DEVICE FOR A HEART SUPPORT SYSTEM

(71) Applicant: KARDION GMBH, Stuttgart (DE)

(72) Inventors: Ingo Stotz, Ditzingen (DE); Fabian Eiberger, Gerlingen (DE)

(21) Appl. No.: 19/265,480

(22) Filed: Jul. 10, 2025

Related U.S. Application Data
(63) Continuation of application No. 17/266,044, filed on Sep. 29, 2021, now Pat. No. 12,390,633.

(30) Foreign Application Priority Data
Aug. 7, 2018 (DE) 102018213150.3

(51) Int. Cl. A61M 60/824; A61M 60/178; A61M 60/183; A61M 60/216; A61M 60/232; A61M 60/806; A61M 60/82; A61M 60/825

(52) U.S. Cl. CPC A61M 60/824; A61M 60/178; A61M 60/183; A61M 60/216; A61M 60/232; A61M 60/806; A61M 60/82; A61M 60/825

(57) ABSTRACT

The disclosure relates to a bearing device for a cardiac support system. The bearing device comprises a stand unit and an impeller. The stand unit is designed to support the impeller such that it can rotate. The impeller is designed to rotate during an operation of the cardiac support system in order to convey a pump fluid flow. The impeller is designed to enclose at least one subsection of the stand unit in the assembled state of the bearing device, wherein an intermediate space for guiding a flushing fluid flow is provided between the subsection and the impeller. At least one flushing outlet is formed in the impeller. The flushing outlet is designed to discharge the flushing fluid flow from the intermediate space by means of centrifugal force when the cardiac support system is in operation.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application is a continuation of U.S. application Ser. No. 17/266,044, filed on Sep. 29, 2021, titled BEARING DEVICE FOR A HEART SUPPORT SYSTEM, AND METHOD FOR RINSING A SPACE IN A BEARING DEVICE FOR A HEART SUPPORT SYSTEM, which is a National Stage Entry of PCT/EP2019/071233, filed on Aug. 7, 2019, titled BEARING DEVICE FOR A HEART SUPPORT SYSTEM, AND METHOD FOR RINSING A SPACE IN A BEARING DEVICE FOR A HEART SUPPORT SYSTEM, which claims priority to German Patent Application No. 10 2018 213 150.3, filed on Aug. 7, 2018, the entire content of which are incorporated by reference herein for all purposes and forms a part of this specification.

### BACKGROUND

### Field

The disclosure relates to a cardiac support system, particularly in flushing an intermediate space of a bearing device of the cardiac support system.

### Description of the Related Art

Cardiac support systems can include pumps and impellers for driving fluid flow through the heart. The cardiac support system can assist a patient's heart in pumping blood through the patient's body by inducing a fluid flow via rotation of the impellers. The cardiac support system generates heat through electrical currents and/or kinetic energy of the rotating impellers. The heat generated by the cardiac support system can increase the risk of thrombosis (blood clots) by dehydrating and thickening the blood. Additionally, cardiac support systems may include cavities or other geometries that can slow or stop blood flow. Stagnation of blood flow may also increase the risk of thrombosis. Accordingly, there remains a need to remove heat from cardiac support systems and mitigate stagnation of blood flow within and around the cardiac support system.

### SUMMARY

The disclosure relates to a bearing device for a cardiac support system comprising a stand unit, an impeller and an intermediate space formed between the impeller and the stand unit for guiding a flushing fluid flow of a fluid, wherein the stand unit comprises a subsection which projects into the impeller and is configured to support the impeller such that it can rotate about an axis of rotation, wherein the impeller is configured to rotate about a longitudinal axis aligned with the axis of rotation when the cardiac support system is in operation to convey a pump fluid flow of the fluid in a flow direction, and wherein the impeller comprises at least one flushing inlet for introducing the flushing fluid flow into the intermediate space and at least one flushing outlet for discharging the flushing fluid flow from the intermediate space.

The invention further relates to a cardiac support system having a bearing device and a method for flushing an intermediate space for guiding a flushing fluid flow with a fluid in a bearing device for a cardiac support system and a method for producing a bearing device for a cardiac support system.

To provide cardiovascular support for patients having heart failure, systems are used in particular that take over part or all of the heart's pumping function. These systems, which are also referred to as cardiac support systems or VADs (ventricular assist devices) for short, can be subdivided into temporary systems for short-term cardiac support and permanent systems for long-term use on or in the patient. One component of such a system is usually a blood pump, typically a centrifugal pump (turbo pump), which is driven by an integrated electric motor and produces the required blood flow by means of a rotor. The pump can be implanted in different locations. The pump can be sutured to the heart from the outside by means of an invasive sternotomy, for example, or it can be placed into the aorta or into a ventricle in a minimally invasive manner by means of a catheter. In the latter case, the maximum permissible outer diameter of the pump is generally limited to 10 mm, which is why the use of an axial pump having a rotor which receives flow axially is desirable. In the process, the blood to be conveyed is expelled through the discharge openings disposed on the circumference of a cylindrical pump housing in order to be returned to the aorta.

EP 3 127 562 A1 discloses a blood pump for a cardiac support system, which comprises a pump housing with an impeller that is rotatably mounted in the pump housing in a sliding bearing having stationary support surfaces, against which support surfaces configured on the blades of the impeller abut. The complex structure of the blades of the impeller on which the support surfaces are formed has the effect that the sliding bearing is flushed and heat is removed from it when blood is pumped in the blood pump.

The object of the disclosure is to provide a bearing device for a cardiac support system that does not require complex blade structures and/or hoses with additional flushing pumps for flushing with a fluid, and to specify a method for flushing a bearing device for a cardiac support system that ensures that sufficient heat can be dissipated from the bearing device during operation of the cardiac support system.

A bearing device according to the disclosure for a cardiac support system includes a stand unit and an impeller and comprises an intermediate space formed between the impeller and the stand unit for guiding a flushing fluid flow of a fluid. The stand unit comprises a subsection which projects into the impeller and is configured to support the impeller such that it can rotate about an axis of rotation. The impeller is configured to rotate about a longitudinal axis aligned with the axis of rotation when the cardiac support system is in operation to convey a pump fluid flow of the fluid in a flow direction, wherein the impeller comprises at least one flushing outlet for discharging the flushing fluid flow from the intermediate space. The at least one flushing outlet in the impeller is configured such that, due to a centrifugal force acting upon the fluid in the at least one flushing outlet, a rotation of the impeller about the axis of rotation during operation of the cardiac support system causes the fluid to be expelled from the intermediate space through the flushing outlet to at least one discharge opening, whereby the flushing fluid flow is discharged from the intermediate space. For this purpose, the at least one flushing outlet in the impeller can comprise a discharge opening for discharging the flushing fluid flow, which has an opening cross-section, in which, at at least one location, an opening cross-section normal vector has a directional component which faces away from the axis of rotation and is radial to the axis of rotation. The at least one flushing outlet in the impeller is configured such that, due to a centrifugal force acting upon the fluid in the at least one flushing outlet, a rotation of the impeller about the axis of rotation during operation of the cardiac support system causes the fluid to be expelled from the intermediate space through the flushing outlet to at least one discharge opening, whereby the flushing fluid flow is discharged from the intermediate space.

A plurality of flushing outlets can be formed in the impeller. The at least one flushing outlet preferably extends along an axis which intersects the longitudinal axis of the impeller or is disposed at an angle to said longitudinal axis. The at least one flushing outlet can in particular be configured as a tube. The at least one discharge opening of the flushing outlet can, for example, be disposed in a jacket section of the impeller enclosing the subsection of the stand unit projecting into the impeller. The at least one discharge opening of the flushing outlet can in particular be disposed in a transition section between a region of a propeller of the impeller and a jacket section of the impeller enclosing the subsection of the stand unit projecting into the impeller.

It is also possible for the impeller to comprise a plurality of flushing outlets, wherein the at least one discharge openings of the flushing outlet are disposed at least partially in a transition section between a region of a propeller of the impeller and a jacket section of the impeller enclosing the subsection of the stand unit projecting into the impeller.

It should be noted that a number of flushing outlets in the impeller can correspond to a multiple of the number of blades of the impeller. It should also be noted that the bearing device can have a flushing inlet which, in the assembled state of the sliding bearing device, opens into the intermediate space. The flushing inlet can be configured as a gap between a base of the stand unit and a jacket section of the impeller enclosing the subsection of the stand unit projecting into the impeller, for example.

It should be noted that the flushing inlet can also be configured as at least one inlet channel extending in a direction which intersects the longitudinal axis of the impeller or extends at an angle to said longitudinal axis. The bearing device can also comprise a flushing inlet having a plurality of inlet channels.

The flushing inlet can in particular be disposed downstream with respect to the flushing outlet in the flow direction of the pump fluid flow.

The impeller can be located in a housing comprising a housing section to which an inlet hose for supplying the fluid is connected.

The housing section of the bearing device preferably has at least one discharge opening for discharging the pump fluid flow. The housing section can comprise webs for connecting to a connection section for connecting an inlet hose, wherein the webs delimit at least one discharge opening of the housing section.

A bearing device according to the disclosure can be configured as a sliding bearing device which comprises a sliding bearing for supporting a rotating component, or as a magnetic bearing device, in which a rotating component is magnetically supported.

A sliding bearing device according to the disclosure comprises a stand unit and an impeller. The stand unit is designed to support the impeller such that it can rotate. The impeller is designed to rotate during an operation of the cardiac support system in order to convey a pump fluid flow. The impeller is configured to enclose at least one subsection of the stand unit in the assembled state of the sliding bearing device. An intermediate space for guiding a flushing fluid flow is provided between said subsection and the impeller. At least one flushing outlet is configured in the impeller to discharge the flushing fluid flow from the intermediate space by means of centrifugal force when the cardiac support system is in operation.

A sliding bearing device according to the disclosure for a cardiac support system in particular enables the sliding bearing device to be flushed by utilizing centrifugal force. For this purpose, an impeller of the sliding bearing device can comprise a flushing outlet that rotates with the impeller in order to use the centrifugal force at the rotating flushing outlet as the driving force for flushing the sliding bearing device. Flushing the sliding bearing device is beneficial during operation of the cardiac support system to dissipate heat and prevent the formation of thromboses.

Flushing that utilizes centrifugal force, as a result of which the flushing rate substantially depends only on the rotational speed of the cardiac support system and not on the static pressure difference between the flushing inlet and the flushing outlet, advantageously reduces the risk of thrombosis formation, because the flushing rate is significantly less affected by loss of pressure in the blood stream and can thus be set more robustly. It is also not necessary for an external pressure difference to be imposed via the flushing system.

The utilization of the centrifugal force via the flushing outlet in the impeller furthermore enables a compact design of the sliding bearing device, which is advantageous in particular for the use of the sliding bearing device in conjunction with the cardiac support system.

The cardiac support system can be a heart pump, for example, such as a left ventricular support system, a right ventricular support system, or a biventricular support system. The stand unit can be understood to be a non-rotating component of the sliding bearing device. The impeller can be a rotating component, such as a rotor. In the assembled state of the sliding bearing device, the impeller can enclose at least one subsection of the stand unit, whereby the sliding bearing device can be configured as a cylindrical sliding bearing, for example. In the implanted state of the cardiac support system, the impeller can be positioned in the blood. The pump fluid flow to be conveyed can, for example, be a blood flow pumped by the cardiac support system and produced by means of the cardiac support system. In the assembled state, an intermediate space in the form of a gap can emerge between the impeller and the subsection of the stand unit. The flushing outlet can be realized as a bore or another type of through-opening in the impeller. The flushing outlet can be configured to conduct the flushing fluid flow from the intermediate space through a portion of the impeller to discharge the flushing fluid flow from the intermediate space. It is also possible to configure two or more flushing outlets in the impeller.

According to one embodiment, the flushing outlet can be inclined relative to a longitudinal axis of the impeller, which in particular corresponds to an axis of rotation of the impeller. This is advantageous for utilizing the centrifugal force to effect a flushing of the sliding bearing device. The flushing outlet can have a longitudinal extension axis which is inclined relative to the longitudinal axis of the impeller. The longitudinal extension axis of the flushing outlet can also be inclined at a right angle with respect to the longitudinal axis of the impeller.

According to one embodiment, the flushing outlet can be configured as a tube having a discharge opening. The flushing outlet can thus advantageously be realized in a cost-saving manner, for example as a bore in the impeller, which also enables a compact design of the sliding bearing device.

According to one embodiment, the discharge opening can be disposed in a jacket section of the impeller enclosing the subsection of the stand unit or in a transition section between a region of a propeller of the impeller and said subsection. The transition section can be configured as a narrowing of the jacket section in the direction of the propeller, for example. The discharge opening can alternatively also be disposed in the region of the propeller. The potential of the centrifugal force, by means of which the flushing effect for flushing the sliding bearing device can advantageously be set, can be set via the positioning of the discharge opening.

According to one embodiment, the impeller can also comprise a plurality of flushing outlets. The discharge openings of the flushing outlets can be disposed at least partially in the transition section. In the assembled state of the sliding bearing device, the flushing outlets can extend radially outward with respect to the stand unit, for example. The discharge openings can be disposed evenly spaced around the periphery of the transition section. This positioning of the flushing outlets and the discharge openings is advantageous in terms of uniform flushing of the intermediate space and in terms of presenting the largest possible cross-section of the flushing outlets.

According to one embodiment, at least one pair of flushing outlets can be configured in the impeller. The flushing outlets of the at least one pair can be disposed opposite one another with respect to a longitudinal axis of the impeller. The configuration of the oppositely disposed pair of flushing outlets is advantageous to prevent an imbalance of the rotating propeller.

A number of flushing outlets in the impeller can correspond to a multiple of the number of blades of the impeller. The flushing outlets in the form of flushing bores are disposed just as periodically as the blading of the impeller, for example. This makes it possible to prevent an imbalance. In this case, for example, two blades result in a multiple of two as the number of flushing outlets.

According to one embodiment, the sliding bearing device can also comprise a flushing inlet for introducing the flushing fluid flow. In the assembled state of the sliding bearing device, the flushing inlet can open into the intermediate space. Using the acting centrifugal force, the flushing fluid flow can flush the intermediate space and thus also the bearing of the sliding bearing device, even without the provision of a static pressure difference between the flushing inlet and the flushing outlet.

According to one embodiment, the flushing inlet can also be configured as a gap between a base of the stand unit and a jacket section of the impeller enclosing the subsection of the stand unit. Additionally or alternatively, the flushing inlet can be configured as an inlet channel in the impeller. The inlet channel can be inclined relative to an axis of rotation of the impeller. The flushing outlet can furthermore be formed in the impeller by a plurality of inlet channels with at least one inclined inlet channel. At least one side of the flushing inlet can thus be configured to be stationary and one side such that it can rotate. The flushing fluid flow can be drawn in on the stationary side of the flushing inlet, e.g., on a wall of the stand unit. If the flushing inlet is configured as an inlet channel in the impeller, the flushing inlet can be configured at least partially in the rotating body of the impeller. A portion of the flushing fluid flow that is partially enclosed in the intermediate space can be introduced through the flushing inlet and discharged again through the flushing outlet, for example to absorb and dissipate heat from the stand unit. The centrifugal pressure is advantageously increased if the flushing inlet is not or only partially located in the rotating body, the impeller.

The flushing inlet can furthermore be disposed downstream with respect to the flushing outlet in the flow direction of the pump fluid flow. By introducing the flushing fluid flow along the stand unit and along the impeller, a constant flushing of the sliding device can advantageously be set thanks to the rotation of the flushing fluid flow at the flushing outlet, even when the pressure levels at the flushing inlet and flushing outlet are the same.

The disclosure further presents a cardiac support system having an embodiment of the aforementioned sliding bearing device. The cardiac support system can be a left ventricular cardiac support pump, for example. For minimally invasive transfemoral or transaortic insertion, for example, the cardiac support system can furthermore have an elongated, cylindrical shape.

A method for producing a bearing device for a cardiac support system configured as a sliding bearing device or as a magnetic bearing device is presented as well. The method comprises the following steps: providing a stand unit, which is designed to support an impeller such that it can rotate, and the impeller, which is configured to rotate during operation of the cardiac support system to convey a pump fluid flow; forming at least one flushing outlet in the impeller, wherein the flushing outlet is designed to discharge a flushing fluid flow from the bearing device by means of centrifugal force when the cardiac support system is in operation; and assembling the impeller and the stand unit to produce the bearing device, wherein at least one subsection of the stand unit is enclosed by the impeller, and wherein an intermediate space for guiding the flushing fluid flow is disposed between the subsection and the impeller.

An embodiment of the aforementioned bearing device can advantageously be produced by carrying out the method.

The condition for the flushing to function by the action of centrifugal force is set out in the following:

The flushing is independent of the static pressure difference. Centrifugal force is used to flush the sliding bearing device; no external pump or additional geometries or structures to produce a static pressure difference are needed. This requires the mechanical energy balance due to the kinetic rotational energy at the exit, at the discharge opening of the flushing outlet, to be positive; i.e., the mechanical energy of the flow at the exit has to be greater than at the entry, at the flushing inlet. This is illustrated in the following using formulas according to Bernoulli's principle:

pₑₓᵢₜ/density−vₑₓᵢₜ²<p_{entry}/density−v_{entry}²/2

If v is the rotational speed and the flushing inlet is not subject to rotation, then:

pₑₓᵢₜ/density−vₑₓᵢₜ^{2/2}<p_{entry}/density

rearranged:

(pₑₓᵢₜ−p_{entry})/density<vₑₓᵢₜ²/2

with the rotation speed v=2 π R n and with n being the speed in revolutions/second

pₑₓᵢₜ−p_{entry}/density<2(πRn)²
from which then follows:
static pressure difference<<2 (πRn)²*density

For water, the “centrifugal pressure” corresponds to a pressure difference of approx. 5 bar at a radius of 1 cm and a speed of 30,000 revolutions/minute. The described approach is therefore effective for this numerical example if the static pressure difference is only approx. 500 mbar (interpreted as “much greater” than a factor of ten).

Flushing of the sliding bearing device by means of centrifugal force requires a rotating system with system limits, the “entry” and “exit”, which point outward in the direction normal to the axis of rotation. The flushing path of the flushing fluid flow extends between the rotating body, the jacket section of the impeller, and the body which is stationary relative to it, the stand unit. According to the design example shown here, the flushing fluid flow moves along the path, i.e., along the intermediate space to the flushing outlet. At the exit of the flushing outlet, the flushing fluid flow flows out of the flushing path. In order to impose the centrifugal force across the entire cross-section, the exit boundary of the flushing outlet is located inside the rotating body, inside the jacket section. The cross-section normal vector should have a component in radial direction, which is not the case on the end face of a cylindrical sliding bearing device, for example, but in radial direction, i.e., when the jacket section is drilled into.

The disclosure also extends to a cardiac support system in which there is a bearing device as described above.

In a method according to the disclosure for flushing an intermediate space for guiding a flushing fluid flow with a fluid in a bearing device for a cardiac support system, wherein the intermediate space comprises at least one flushing inlet for introducing the flushing fluid flow and at least one flushing outlet for discharging the flushing fluid flow and wherein the intermediate space is configured between an impeller which can rotate about an axis of rotation for conveying a pump fluid flow and a stand unit for rotatably supporting the impeller, in which the fluid is introduced into the intermediate space through the at least one flushing inlet, the fluid is expelled from the intermediate space through the flushing outlet to at least one discharge opening by means of a centrifugal force acting upon said fluid in the at least one flushing outlet relative to the axis of rotation.

Advantageous design examples of the disclosure are described in more detail in the following with reference to schematic drawings.

### BRIEF DESCRIPTION OF THE DRAWINGS

The figures show:

FIG. 1 a first sliding bearing device for a cardiac support system comprising an impeller and comprising a stand unit as a section;

bearing device;

FIG. 2 a portion of a cardiac support system comprising the first sliding

FIG. 3 a side view of the first sliding bearing device;

FIG. 4 a rear view of the impeller in the direction of the arrow IV of FIG. 3;

FIG. 5 other possible designs of an impeller in a sliding bearing device for a cardiac support system;

FIG. 6 an intermediate space having various flushing fluid volumes in different sliding bearing devices for a cardiac support system with different configurations of flushing outlets;

FIG. 7 a further sliding bearing device comprising an impeller and comprising a stand unit;

FIG. 8 the further sliding bearing device comprising an impeller and comprising a stand unit as a section;

FIG. 9 a detail of a further sliding bearing device for a cardiac support system in a sectional view;

FIG. 10 the detail of the further sliding bearing device for a cardiac support system of FIG. 9 in a plan view;

FIG. 11 a detail of a further sliding bearing device for a cardiac support system in a sectional view; and

FIG. 12 a flow diagram of a method for producing a sliding bearing device.

### DETAILED DESCRIPTION

In the following description of favorable design examples of the present disclosure, the same reference signs are used for the elements shown in the various figures, which are the same or have a similar effect, whereby a repeated description of these elements is omitted.

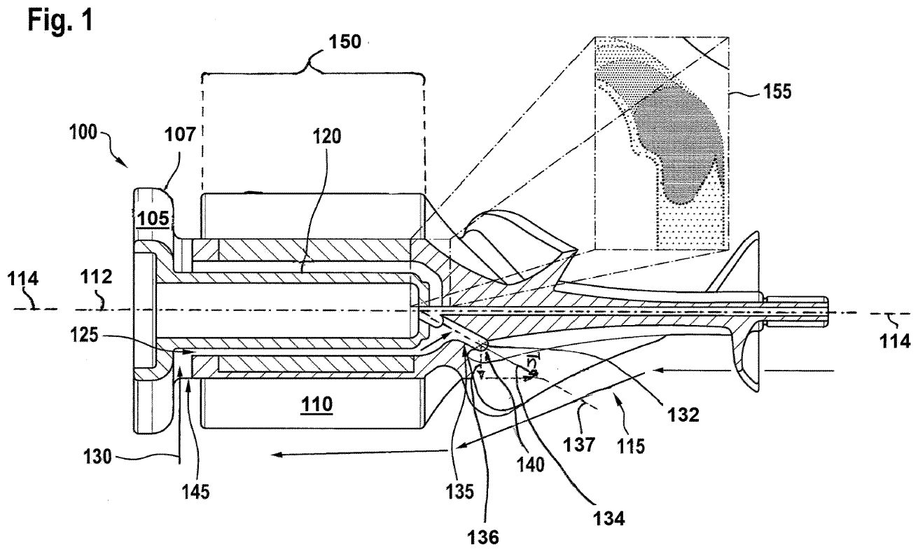

FIG. 1 shows a schematic illustration of a bearing device 100 for a cardiac support system which is configured as a sliding bearing device according to one design example. The bearing device 100 comprises a stand unit 105 and an impeller 110. The stand unit 105 is configured to support the impeller 110 such that it can rotate about an axis of rotation 112 which is coaxial with the longitudinal axis 114 of the impeller 110. The impeller 110 is designed to rotate about the axis of rotation 112 when the cardiac support system is in operation in order to convey a pump fluid flow 115. In the assembled state of the sliding bearing device shown here, the impeller 110 encloses at least one subsection 120 of the stand unit 105. An intermediate space 125 for guiding a flushing fluid flow 130 is provided between the subsection 120 and the impeller 110. At least one flushing outlet 135 is formed in the impeller 110. The flushing outlet 135 is designed to discharge the flushing fluid flow 130 from the intermediate space 125 by means of centrifugal force when the cardiac support system is in operation.

The flushing outlet 135 comprises a discharge opening 140 for discharging the flushing fluid flow 130, which has an opening cross-section 132, in which, at at least one location, an opening cross-section normal vector 134 has a directional component 136 which faces away from the axis of rotation 112 and is radial to the axis of rotation 112.

According to the design example shown here, the flushing outlet 135 is inclined relative to the longitudinal axis 114 of the impeller 110 which is coaxial with the axis of rotation 112. The flushing outlet 135 comprises an axis 137 along which said flushing outlet 135 extends and which is thus a longitudinal extension axis of the flushing outlet 135, which is inclined relative to the longitudinal axis 114 of the impeller 110 and forms an acute angle α with it. It should be noted that this axis 137 can also be inclined relative to the longitudinal axis 114 of the impeller 110.

Furthermore, according to the design example shown here, the flushing outlet 135 is configured as a tube with a discharge opening 140. The discharge opening 140 is disposed at an end of the tube facing away from the intermediate space 125.

According to the design example shown here, the sliding bearing device 100 also comprises a flushing inlet 145 for introducing the flushing fluid flow 130. In the assembled state of the bearing device 100 shown here, the flushing inlet 145 opens into the intermediate space 125.

According to the design example shown here, the flushing inlet 145 is configured as a gap between a base 107 of the stand unit 105 and a jacket section 150 of the impeller 110 enclosing the subsection 120 of the stand unit 105. It should be noted that the flushing inlet can in principle also be configured as an inlet channel in the impeller 110.

In the sliding bearing device shown in FIG. 1, the flushing inlet 145 is disposed downstream with respect to the flushing outlet 135 in the flow direction of the pump fluid flow 115 as in the design example shown here. FIG. 1 shows a flushing fluid flow 130 with a flushing path for flushing the bearing device 100 which extends from the flushing inlet 145 through the intermediate space 125 to the flushing outlet 135 with the discharge opening 140.

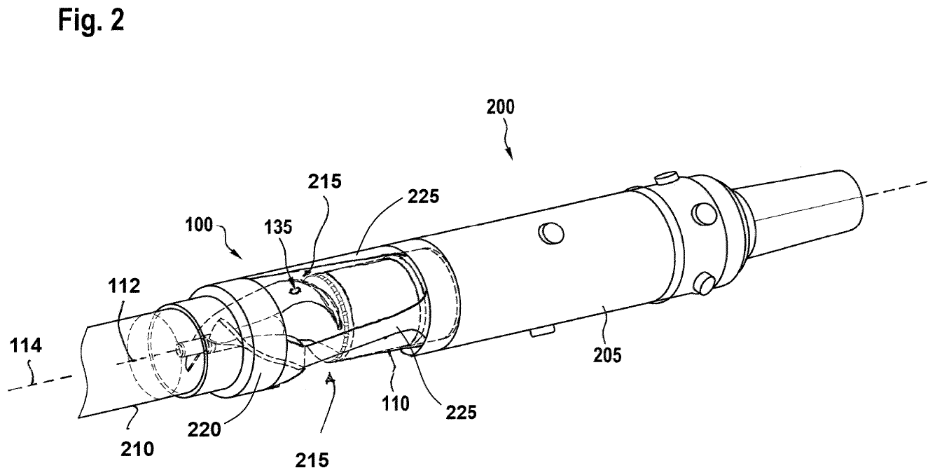

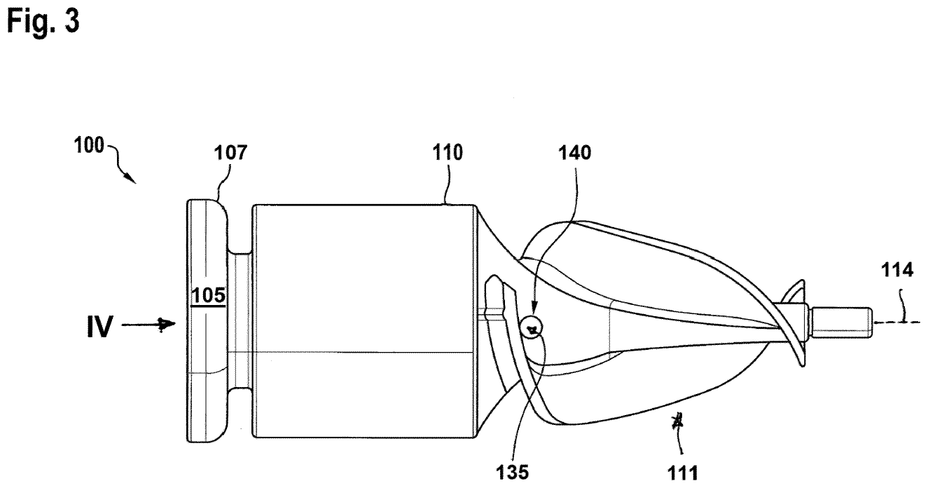

FIG. 2 shows a perspective view of a portion of a cardiac support system 200 with the sliding bearing device 100 in the form of a left ventricular cardiac support pump (LVAD heart pump). FIG. 3 is a side view of the bearing device 100.

The bearing device 100 and its function in a cardiac support system are described in more detail in the following:

The impeller 110 is a rotor that forms a rotating component in the bearing device 100 of the cardiac support system 200, which is supported magnetically or by means of sliding bearings, wherein the rotating component is positioned over a fluid to dissipate heat or reduce friction. When the impeller 110 is positioned directly in the blood during operation of the cardiac support system, as is the case, for example, with the left ventricular cardiac support pump (LVAD heart pump) shown in FIG. 2 in the implanted state of the cardiac support system, it is beneficial to flush the bearing device 100 to dissipate heat and prevent the formation of thromboses (“blood clotting”). To enable robust flushing of the sliding bearing device 100, a constant flow is necessary. Flushing the sliding bearing device 100 prevents the formation of thromboses. A pump design (such as baffles) that converts mechanical energy into hydrodynamic energy can be used for this purpose. With the sliding bearing device 100 shown in FIG. 1 and FIG. 2, it is possible to utilize the centrifugal force on the flushing outlet 135 rotating with the impeller 110 using only a bore in the form of the flushing outlet 135. The centrifugal force represents the driving force for the flushing. Such a structure is inexpensive to produce.

A plurality of flushing outlets 135 can alternatively also be provided at different locations on the impeller 110 to utilize the centrifugal force, as shown in the following figures.

Using a design example of the bearing device 100 shown here, introduction can be realized by suctioning out the flushing fluid flow 130 with the aid of the centrifugal force at the flushing outlet 135. Structurally, this is achieved by configuring the flushing outlet 135 such that the flushing outlet 135 is enclosed by the rotating component, the impeller 110, e.g., by having a bore as the flushing outlet 135, while the inlet side in the form of flushing inlet 145 is not or only partially, e.g., only on one side, subject to the rotation. This is achieved by configuring the flushing inlet 145 with at least one section of the stand unit 105 as a wall section. In this case, the statistical pressure difference has practically no effect on the flushing flow of the flushing fluid flow 130, which is why the flushing effect of the bearing device 100 is substantially determined by the centrifugal force and the rotational speed of the pump of the cardiac support system. The flushing effect of the bearing device 100 is thus largely independent of other potential influencing variables, such as the magnitude of the mass flow or the level of the pressure build-up through or over the cardiac support system. Consequently, there is no need for a static pressure difference to flush the bearing device 100. The positioning of the flushing outlet 135 in the impeller 110, which is trumpet-shaped here as an example, with widely varying diameters relative to a longitudinal extension axis 114 of said impeller 110, can therefore be realized in different ways, whereby a positioning of the flushing outlet 135 far upstream of the longitudinal extension of the impeller 110 can be omitted. Complex structures, such as a pump wheel, or the application of a pressure difference in or around the sliding bearing device 100 are not necessary to effect the flushing of the sliding bearing device 100 either. Because of the independence from the pump flow, the pump flow of the pump fluid flow 115 shown here, the flushing of the bearing device 100 is possible without an absence of flushing as long as the impeller 110 is rotating.

In the design example discussed here, the bearing device 100 comprises the impeller 110 as a rotating part which, together with the stand unit 105 as a stationary part, forms a cylindrical sliding bearing. The flushing effect of the bearing device 100 is based on the centrifugal force that results from a rotation at the flushing outlet 135. The prerequisite for this is that, as shown here, at least one side at the flushing inlet 145 is stationary; in this case the inner side in the form of the stand unit 105. As a result, even if the pressure levels at the flushing inlet 145 and the flushing outlet 135 are comparable or the same, a constant flushing of the sliding bearing device 100 can be set due to the rotation of both sides of the flushing outlet 135 formed in the rotating impeller 110 or the fluid volume of the flushing outlet 135. The design example of the bearing device 100 shown here also makes it possible to flush a partially enclosed volume, which is shown here in block 155 which, as an example, is disposed around the fixed bearing of the stand unit 105, by combining a rotating and a stationary side. The reason for this is that the flushing fluid flow 130 is accelerated on the rotating side of the impeller as a result of the molecular adhesion conditions. The flushing fluid flow 130 is accelerated along the wall of the intermediate space 125 toward a larger diameter due to the centrifugal force, as a result of which the flushing fluid flow 130 is drawn in on the stationary side of the intermediate space 125 in the form of a wall of the stand unit 105. This causes the partially enclosed fluid of the flushing fluid flow 130 to be flushed, which allows heat at the fixed bearing of the stand unit 105, for example, to be absorbed and dissipated.

The cardiac support system 200 shown in FIG. 2 comprises a housing section 205. The impeller 110 of the bearing device 100 is located in the housing section 205 of the cardiac support system 200. In the cardiac support system 200, the impeller 110 is disposed in a housing section 205 on which an inlet hose 210 for supplying the fluid is provided. In the housing section 205 of the housing of the cardiac support system, there are discharge openings 215 for discharging the pump fluid flow 115. For connecting the inlet hose 210, the cardiac support system 200 comprises a connection section 220 which is connected to webs 225 of the housing section 205 that delimit two discharge openings 215 for discharging fluid conveyed by a rotation of the impeller 110 in the cardiac support system 200 from the housing section 205.

The housing section 205 of the cardiac support system 200 has a cylindrical, elongated structure with a substantially constant outer diameter for easy placement in a blood vessel, such as the aorta, by means of a catheter. The elongated axial design shown here allows transfemoral implantation of the cardiac support system 200. The sliding bearing device 100 is accordingly disposed in a window opening in the housing section 205 such that, in the implanted state of the cardiac support system 200, the rotating rotor component, the impeller 110, is positioned in the blood. Due to the axial design of the cardiac support system 200, the flow received by the impeller 110 is axial relative to the longitudinal axis 114 of the impeller 110, which corresponds to a longitudinal axis of the cardiac support system 200. The flushing outlet 135 in the impeller 110 is disposed in the region 111 of a propeller of the impeller 110, whereby the flushing outlet 135 is realized by a drilled hole or a through-bore or another type of through-hole in the impeller 110.

FIG. 3 shows the sliding bearing device 100 with the stand unit 105 and the impeller 110 in the assembled state, whereby the stand unit 105 forms the non-rotating counterpart to the rotating impeller 110. The stand unit 105 has a section which narrows in the direction of the impeller 110. The narrowed section of the stand unit 105 is mostly enclosed by the impeller 110. The stand unit 105 is connected to the impeller 110 and supports the impeller 110 such that it can rotate. The flushing outlet 135 having a discharge opening 140 is configured in the impeller 105. As an example, the discharge opening 140 of the flushing outlet here is disposed in the region of the propeller of the impeller 110.

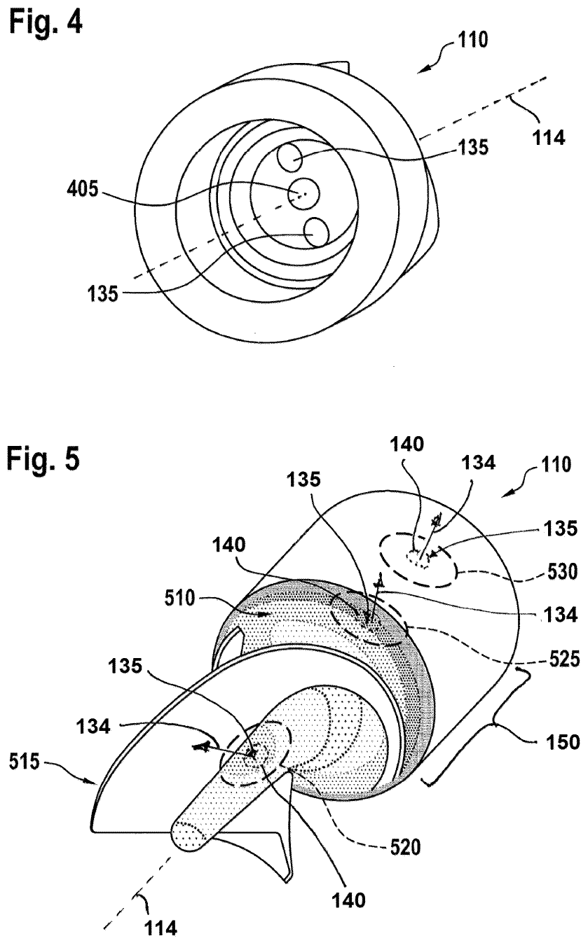

FIG. 4 shows a perspective rear view of the impeller in the direction of the arrow IV of FIG. 3. The side of the impeller 110 facing away from the propeller of the impeller 110, which can be coupled to the stand unit 105 of the bearing device, is shown as the rear side of the impeller 110. To connect the impeller 110 to the stand unit 105, the impeller 110 here comprises a ball bearing 405 for supporting the impeller 110. The flushing outlets 135 of the impeller 110, which, as an example, are configured here as discharge bores and which communicate with the intermediate space 125 shown in FIG. 1, can be seen as well.

According to the design example shown here, at least one pair of flushing outlets 135 is configured in the impeller 110. The flushing outlets 135 of the at least one pair are disposed opposite one another with respect to a longitudinal axis 114 of the impeller 110. As an example, the flushing outlets 135 of the pair are evenly spaced with respect to the axis of rotation 112 of the impeller 110, i.e., they extend symmetrically relative to a longitudinal axis 114 of the impeller 110 coaxial with the axis of rotation 112.

FIG. 5 shows further possible designs of an impeller 110 in a bearing device for a cardiac support system, which can be configured as a sliding bearing device or as a magnetic bearing device. The figure shows a perspective view of the impeller 110, wherein different example positionings of a discharge opening 140 of the flushing outlet 135 in the impeller 110 as well as a respective opening cross-section normal vector 134 and the longitudinal axis 114 of the impeller 110 are identified.

According to one design example, the discharge opening 140 of the flushing outlet 135 is disposed in a jacket section 150 of the impeller 110 enclosing the subsection of the stand unit. Alternatively, the discharge opening of the flushing outlet is disposed in a transition section 510 between a region of a propeller 515 of the impeller 110 and the jacket section 505.

This figure shows a potential estimate for the design example, where the strongest suction force occurs, and thus where a suitable location for positioning the flushing outlet and the discharge opening of the flushing outlet is. Three regions 520, 525 and 530 for disposing the discharge opening of the flushing outlet in the impeller 110 are shown as examples. The region 520 is located in the region of the propeller 515. The region 525, for example, identifies a position of the discharge opening of the flushing outlet 135 in the transition section 510. The region 530, for example, identifies a positioning of the discharge opening of the flushing outlet in the jacket section 150. According to the potential estimate shown here, when the flushing outlet 135 and the discharge opening 140 are positioned in the region 530, a beneficial flushing effect is achieved in a bearing device having such an impeller 110 and a stand unit 105 because the centrifugal force between the flushing inlet and the flushing outlet is sufficient to drive the flushing.

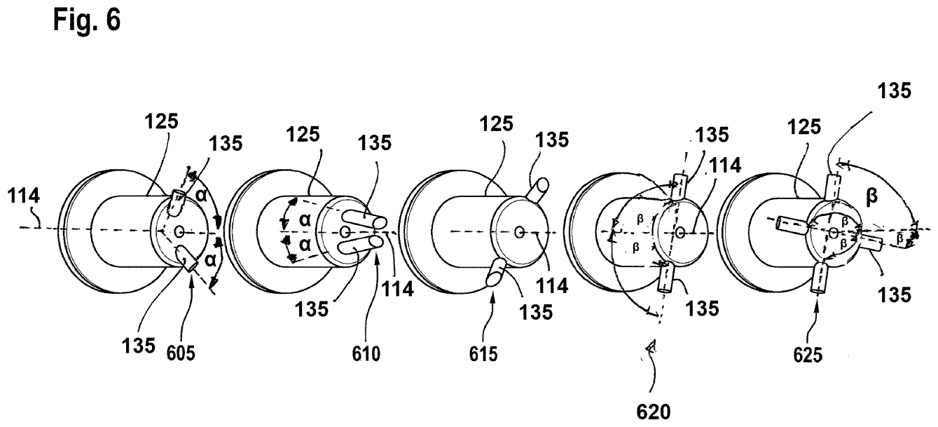

FIG. 6 shows the intermediate space 125 with different flushing fluid volumes in different bearing devices for a cardiac support system designed as a sliding bearing device or as a magnetic bearing device having different configurations of flushing outlets, wherein the flushing outlets 135 are configured differently. The flushing outlet 135, through which the flushing fluid flow passes, has different configurations 605, 610, 615, 620, 625 here. At least one pair of flushing outlets 135 is configured in the impeller of these sliding bearing devices, whereby the flushing outlets 135 of the at least one pair in a bearing device are disposed opposite one another with respect to the longitudinal axis 114 of the impeller 110 aligned with the axis of rotation 112. The respective configurations 605, 610, 615, 620, 625 of the flushing outlets shown here show examples of the pair of flushing outlets. In a first configuration 605, the flushing outlets of the pair extend radially from the longitudinal axis 114 of the impeller inclined at an obtuse angle α with respect to said longitudinal axis 114 of the impeller, whereby a starting point of the flushing outlets 135 is formed in close proximity to the longitudinal axis 114. In a second configuration 610, the flushing outlets 135 of the pair extend inclined at an acute angle α with respect to the longitudinal axis 114 of the impeller; the flushing outlets 135 of the pair are accordingly angled toward one another. A third configuration 615 corresponds to the first configuration 605 with the exception of the starting point of the flushing outlets 135, which are disposed further apart than the starting points of the flushing outlets of the first configuration 605. In a fourth configuration 620, the flushing outlets 135 of the pair extend at a right angle β to the longitudinal axis 114 of the impeller. A fifth configuration 625 shows an example of two pairs of flushing outlets 135, which are disposed opposite one another with respect to the longitudinal axis 114 of the impeller and are disposed evenly spaced apart from one another. Like the pair shown in the fourth configuration 620, the two pairs of flushing outlets 135 extend at a right angle ß to the longitudinal axis 114 of the impeller.

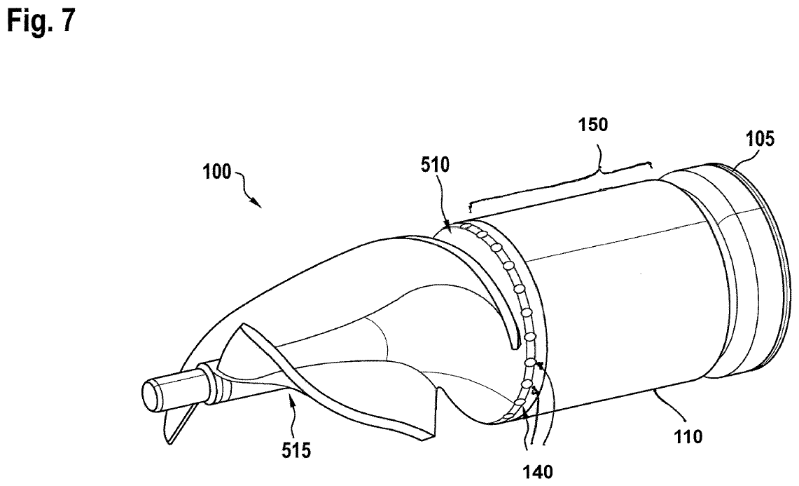

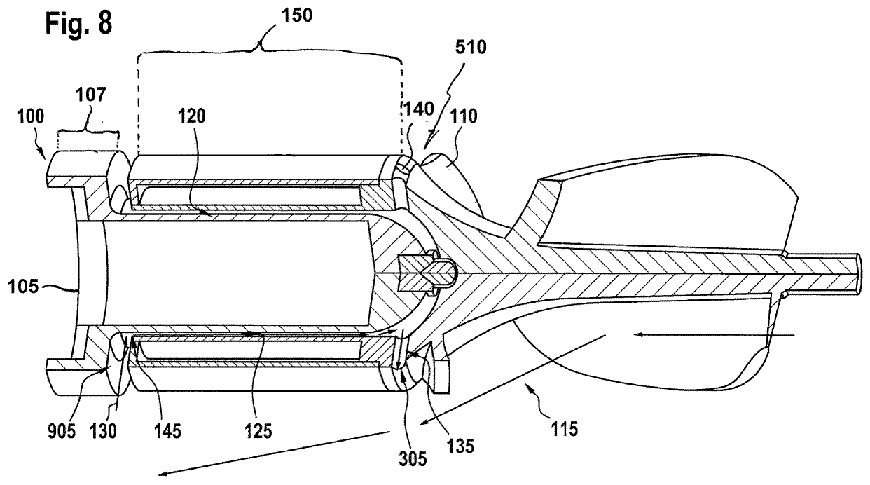

FIG. 7 shows a further sliding bearing device 100 for a cardiac support system. The figure shows a perspective view of the sliding bearing device 100 in the assembled state, in which the impeller partly encloses the stand unit 105. FIG. 8 shows this sliding bearing device 100 as a section. The sliding bearing device 100 shown here is similar to the sliding bearing device described with reference to the preceding figures. According to the design example shown here, the impeller 110 comprises a plurality of flushing outlets 135. The discharge openings 140 of the flushing outlets are disposed at least partially in the transition section 510 between the propeller 515 and the jacket section 505. As an example, the discharge openings 140 are disposed evenly spaced around the periphery of the transition section 510. FIG. 7 shows a utilization of the flushing position of the plurality of flushing outlets having the suction force determined to be the strongest.

FIG. 8 shows a further sliding bearing device 100 for a cardiac support system. The figure shows a sectional view of a side view of the sliding bearing device 100. The stand unit 105 is partially enclosed by the jacket section 150 of the impeller 110. The plurality of discharge openings 140 of flushing outlets 135 is disposed in the transition region or transition section 510 between the propeller of the impeller 110 and the jacket section 150. The figure shows the flow direction of the pump fluid flow 115 and the flow path of the flushing fluid flow 130. The flushing fluid flow 130 is introduced through the flushing inlet 145 which, according to the design example shown here, is configured as a gap 905 between the base 107 of the stand unit 105 and the jacket section 505 of the impeller 110 enclosing the subsection 120 of the stand unit 105. The flushing fluid flow 130 is then conducted through the intermediate space 125 to one of the discharge openings 140 of the plurality of flushing outlets 135 by means of centrifugal force in order to flush the sliding bearing device 100.

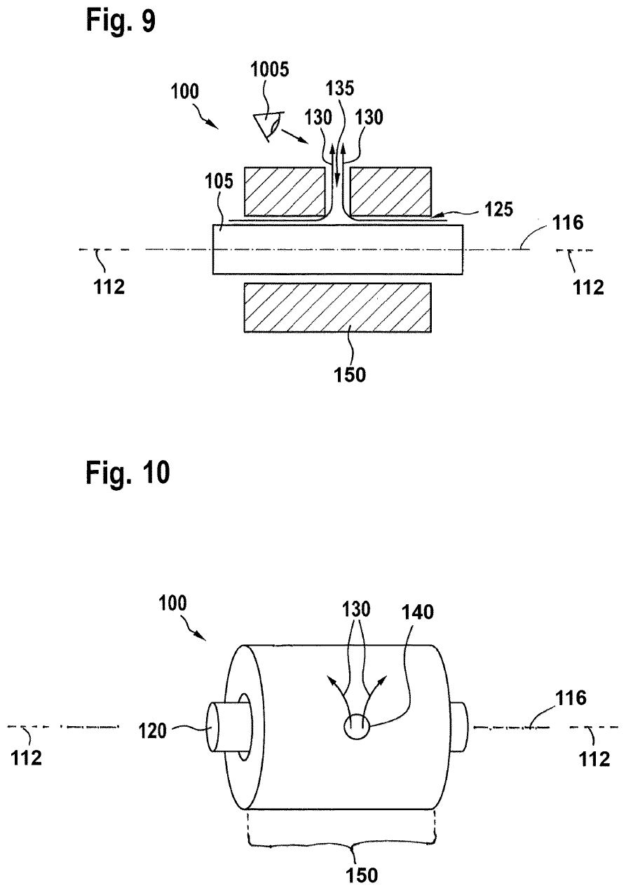

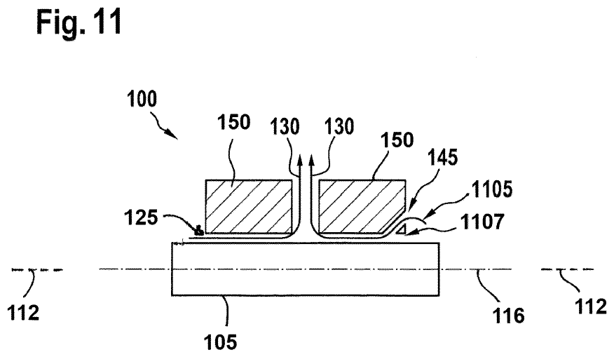

FIG. 9 shows a schematic illustration of a detail of a sliding bearing device 100 for a cardiac support system according to one design example. The figure shows a cross-section of a part of the sliding bearing device 100 with the subsection of the stand unit 105 enclosed by the jacket section 150 of the impeller. The configuration of the flushing outlet 135 here is intended to show that the flushing outlets can also be disposed in a non-mirror-symmetrical manner. The figure shows a portion of the flushing path of the flushing fluid flow 130 that flows through the intermediate space 125 to the flushing outlet 135 and is discharged from the discharge opening of the flushing outlet 135. The outflow of the flushing fluid flow is shown in the following FIG. 10 with the aid of a plan view from the direction identified here with the arrow 1005.

FIG. 10 shows a schematic illustration of a detail of a sliding bearing device 100 for a cardiac support system according to one design example. The figure shows a plan view onto the detail of the sliding bearing device 100 identified in the preceding FIG. 9. The flushing outlet 135 is disposed in the jacket section 150 radially to a longitudinal extension axis 116 of the subsection of the stand unit 105 enclosed by the jacket section 505 which is aligned with the axis of rotation 112 in the bearing device. The flushing fluid flow 130 exits the jacket section 150 at the discharge opening 140 of the flushing outlet.

FIG. 11 shows a schematic illustration of a detail of a sliding bearing device 100 for a cardiac support system according to one design example. According to the design example shown here, the flushing inlet 145 is realized in the intermediate space 125 by a plurality of inlet channels, namely by the channel 1105 and the channel 1107. This is also intended to demonstrate that the inlet direction does not necessarily only have to be oriented in the direction of the longitudinal extension axis 116 of the bearing device 100 aligned with the axis of rotation 112 of the bearing device 100, but can also be inclined relative to said longitudinal extension axis. If the flushing inlet 145 is configured such that there is no acting centrifugal force there, for example such that the boundary of the flushing inlet 145 is not or only partially in the rotating body as in the design example of the flushing inlet 145 shown here as an inlet channel 1105 which is partially configured in the jacket section 150, the centrifugal pressure is advantageously increased.

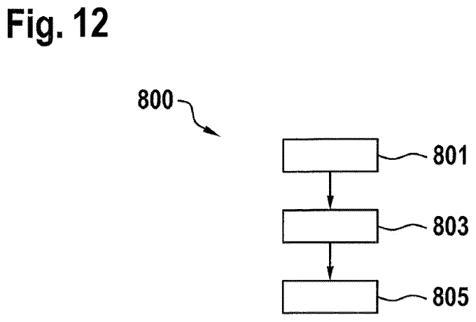

FIG. 12 shows a flow diagram of a method 800 for producing a bearing device for a cardiac support system configured as a sliding bearing device or as a magnetic bearing device according to one design example. The method 800 comprises a step 801 of providing, a step 803 of forming, and a step 805 of assembling. In step 801 of providing, a stand unit is provided, which is configured to support an impeller such that it can rotate. Also provided in step 801 is the impeller, which is designed to rotate during an operation of the cardiac support system in order to convey a pump fluid flow. In step 803 of forming, at least one flushing outlet is formed in the impeller, which is designed to discharge a flushing fluid flow from the sliding bearing device by means of centrifugal force when the cardiac support system is in operation. In step 805 of assembling, the impeller and the stand unit are assembled to produce the sliding bearing device. At least one subsection of the stand unit is enclosed by the impeller. An intermediate space for guiding the flushing fluid flow is furthermore provided between said subsection and the impeller. During the operation of the cardiac support system, the flushing fluid flow is conducted from the intermediate space into the flushing outlet by means of centrifugal force and from there is discharged from the bearing device in order to flush the bearing device.

In summary, in particular the following should be noted: The disclosure relates to a bearing device 100 for a cardiac support system. The bearing device 100 comprises a stand unit 105 and an impeller 110. The stand unit 105 is designed to support the impeller 110 such that it can rotate. The impeller 110 is designed to rotate when the cardiac support system is in operation in order to convey a pump fluid flow 115. The impeller 110 is designed to enclose at least one subsection 120 of the stand unit 105 in the assembled state of the bearing device 100, wherein an intermediate space 125 for guiding a flushing fluid flow 130 is provided between the subsection 120 and the impeller 110. At least one flushing outlet 135 is formed in the impeller 110. The flushing outlet 135 is designed to discharge the flushing fluid flow 130 from the intermediate space 125 by means of centrifugal force when the cardiac support system is in operation.

The disclosure relates, in particular, to the aspects specified in the following clauses:

Clause 1. Sliding bearing device (100) for a cardiac support system (200), wherein the sliding bearing device (100) has the following features:
a stand unit (105) is designed to support an impeller (110) such that it can rotate; and
the impeller (110), which is configured to rotate when the cardiac support system (200) is in operation to convey a pump fluid flow (115), wherein the impeller (110) is designed to enclose at least one subsection (120) of the stand unit (105) in the assembled state of the sliding bearing device (100), wherein an intermediate space (125) for guiding a flushing fluid flow (130) is provided between the subsection (120) and the impeller (110), wherein at least one flushing outlet (135) is formed in the impeller (110), wherein the flushing outlet (135) is designed to discharge the flushing fluid flow (130) from the intermediate space (125) by means of centrifugal force when the cardiac support system (200) is in operation.

Clause 2. Sliding bearing device (100) according to clause 1, wherein a plurality of flushing outlets (135) are formed in the impeller (110).

Clause 3. Sliding bearing device (100) according to any one of the preceding clauses, wherein the at least one flushing outlet (135) is inclined relative to a longitudinal axis of the impeller (110).

Clause 4. Sliding bearing device (100) according to any one of the preceding clauses, wherein the flushing outlet (135) is configured as a tube having a discharge opening (140).

Clause 5. Sliding bearing device (100) according to clause 4, wherein the discharge opening (140) is disposed in a jacket section (505) of the impeller (110) enclosing the subsection (120) of the stand unit (105) or in a transition section (510) between a region of a propeller (515) of the impeller (110) and the jacket section (150).

Clause 6. Sliding bearing device (100) according to clause 5, wherein the impeller (110) comprises a plurality of flushing outlets (135), wherein the discharge openings (140) of the flushing outlets (135) are at least partially disposed in the transition section (510).

Clause 7. Sliding bearing device (100) according to any one of the preceding clauses, wherein a number of the flushing outlets (135) in the impeller (110) corresponds to a multiple of the number of blades of the impeller (110).

Clause 8. Sliding bearing device (100) according to any one of the preceding clauses, comprising a flushing inlet (145) for introducing the flushing fluid flow (130), wherein, in the assembled state of the sliding bearing device (100), the flushing inlet (145) opens into the intermediate space (125).

Clause 9. Sliding bearing device (100) according to clause 8, wherein the flushing inlet (145) is formed as a gap (905) between a base of the stand unit (105) and a jacket section (150) of the impeller (110) enclosing the subsection (120) of the stand unit (105), and/or wherein the flushing inlet (145) is formed in the impeller (110) as an inclined inlet channel (1105) or by a plurality of inlet channels having at least one inclined inlet channel (1105).

Clause 10. Sliding bearing device (100) according to any one of clauses 8 to 9, wherein the flushing inlet (145) is disposed downstream with respect to the flushing outlet (135) in the flow direction of the pump fluid flow (115).

Clause 11. Cardiac support system (200) comprising a sliding bearing device (100) according to any one of the preceding clauses 1 to 10.

Clause 12. Method (800) for producing a sliding bearing device (100) for a cardiac support system (200), wherein the method (800) comprises the following steps:
providing (801) a stand unit (105), which is designed to support an impeller (110) such
   that it can rotate, and the impeller (110), which is configured to rotate during operation of the cardiac support system (200) to convey a pump fluid flow (115);
forming (803) at least one flushing outlet (135) in the impeller (110), wherein the flushing outlet (135) is designed to discharge a flushing fluid flow (130) from the sliding bearing device (100) by means of centrifugal force when the cardiac support system (200) is in operation; and
assembling (805) the impeller (110) and the stand unit (105) to produce the sliding bearing device (100), wherein at least one subsection (120) of the stand unit (105) is enclosed by the impeller (110), and wherein an intermediate space (125) for guiding the flushing fluid flow (130) is disposed between the subsection (120) and the impeller (110).

### LIST OF REFERENCE SIGNS

100 Sliding bearing device
105 Stand unit
107 Base
110 Impeller
111 Region of a propeller of the impeller
112 Axis of rotation
114 Longitudinal axis
115 Pump fluid flow
116 Longitudinal extension axis
120 Subsection of the stand unit
125 Intermediate space
130 Flushing fluid flow
132 Opening cross-section
134 Opening cross-section normal vector
135 Flushing outlet
136 Directional component
137 Axis
140 Discharge opening
145 Flushing inlet
150 Jacket section
155 Block
200 Cardiac support system
205 Housing section
210 Inlet hose
215 Discharge opening
220 Connection section
225 Web
405 Ball bearing
505 Jacket section
510 Transition section
515 Propeller
520, 525, 530 Region
605, 610, 615, 620, 625 Configuration
800 Method
801 Step of providing
803 Step of forming
805 Step of assembling
905 Gap
1005 Arrow
1105, 1107 Inlet channel

What is claimed is:

1. A heart pump comprising:
a housing comprising one or more discharge openings;
a support structure positioned within the housing, the support structure disposed along an axis of rotation;
an impeller positioned radially outward from at least a portion of the support structure and configured to rotate about the axis of rotation, the impeller comprising one or more flushing outlets each having a longitudinal axis extending away from the support structure and parallel to a plane orthogonal to the axis of rotation, wherein the longitudinal axes of the flushing outlets are configured to intersect the discharge openings of the housing as the impeller rotates about the axis of rotation; and
an intermediate space formed between the impeller and the portion of the support structure.

2. The heart pump of claim 1, wherein a flushing flow is induced by the rotation of the impeller about the axis of rotation, the flushing flow configured to enter the intermediate space at an inlet positioned between the impeller and the support structure and to exit the intermediate space through the flushing outlets.

3. The heart pump of claim 2, wherein the flushing flow exits the heart pump through the discharge openings of the housing after exiting the flushing outlets of the impeller.

4. The heart pump of claim 1, wherein the housing further comprises a hollow cylindrical body, wherein the discharge openings extend through a wall of the hollow cylindrical body.

5. The heart pump of claim 1, wherein the impeller comprises a jacket section, wherein the jacket section axially overlaps the portion of the support structure.

6. The heart pump of claim 5, wherein the jacket section is a hollow cylindrical body having an inner surface and an outer surface positioned radially outward from the inner surface.

7. The heart pump of claim 5, wherein the jacket section of the impeller is parallel with the portion of the support structure.

8. The heart pump of claim 5, wherein the one or more flushing outlets are positioned in the jacket section.

9. The heart pump of claim 5, wherein the impeller further comprises a region of a propeller and a transition section positioned between the region of the propeller and the jacket section.

10. The heart pump of claim 9, wherein the one or more flushing outlets are positioned in the transition section.

11. The heart pump of claim 9, wherein the one or more flushing outlets are positioned in the region of the propeller.

12. The heart pump of claim 1, wherein the intermediate space extends between an outer surface of the portion of the support structure and an inner surface of the impeller.

13. A heart pump comprising:
a housing comprising one or more discharge openings;
a support structure positioned within the housing, the support structure disposed along an axis of rotation;
an impeller positioned radially outward from at least a portion of the support structure and configured to rotate about the axis of rotation, the impeller comprising one or more flushing outlets each having a longitudinal axis extending away from the support structure and parallel to a plane orthogonal to the axis of rotation, wherein the longitudinal axes of the flushing outlets are configured to intersect the discharge openings of the housing as the impeller rotates about the axis of rotation; and
a fluid flow path extending from a flushing inlet positioned between an end of the impeller and the portion of a support structure to the discharge openings through the flushing outlets.

14. The heart pump of claim 13, wherein a flushing flow is induced by the rotation of the impeller about the axis of rotation.

15. The heart pump of claim 13, wherein the impeller comprises a jacket section, a region of a propeller, and a transition section positioned between the region of the propeller and the jacket section, wherein the jacket section axially overlaps the portion of the support structure.

16. The heart pump of claim 15, wherein the one or more flushing outlets are positioned in the transition section.

17. The heart pump of claim 15, wherein the one or more flushing outlets are positioned in the jacket section.

18. The heart pump of claim 13, wherein the flushing inlet comprises a longitudinal axis.

19. The heart pump of claim 18, wherein the longitudinal axis of the flushing inlet is parallel to the axis of rotation.

20. The heart pump of claim 18, wherein the longitudinal axis of the flushing inlet intersects the discharge openings of the housing.
